(19) United States
(12) Patent Application Publication
(10) Pub. No.: US 2016/0199229 A1
(43) Pub. Date: Jul. 14, 2016

(54) Impregnable and Expanding Wound Packing Article and Method of Use

(71) Applicant: Terence W. Kolb, Fletcher, NC (US)

(72) Inventor: Terence W. Kolb, Fletcher, NC (US)

(21) Appl. No.: 15/074,153

(22) Filed: Mar. 18, 2016

Related U.S. Application Data
(63) Continuation-in-part of application No. 14/978,181, filed on Dec. 22, 2015.
(60) Provisional application No. 62/095,216, filed on Dec. 22, 2014.

(51) Int. Cl. A61F 13/00

(52) U.S. Cl. CPC A61F 13/00068; A61F 13/00021; A61F 13/00012; A61F 13/00017; A61F 13/00063; A61F 2013/00357

(57) ABSTRACT

An impregnable and expanding wound packing article includes a main body at least partially formed from a highly absorbent material, such as polyurethane sponge material or compressed cotton gauze sponge material. In response to absorption of surrounding fluids, the main body continually expands until the outer wall of the main body comes into abutment with the inner wall of the irrigated, open wound. An elongated piece of material forming a wick extends from the main body. In one embodiment, the wick defines an evacuation channel extending between an opening at a distal end of said wick and a proximal end of said wick located within said main body, the wick being at least partially absorbent for permitting passage therethrough of the purulent fluid, and wherein the purulent fluid may be accessed and removed via said evacuation channel.

This application is a continuation-in-part application of co-pending patent application Ser. No. 14/978,181 filed on Dec. 22, 2015, which is based on and claims priority to provisional patent application No. 62/095,216 filed on Dec. 22, 2014.

### BACKGROUND OF THE INVENTION

1. Field of the Invention

This invention relates to treatment of open wounds, such as abscesses, and, more particularly, to an impregnable and expanding article for packing open wounds.

2. Discussion of the Related Art

Presently used treatment methods for packing wounds have been in place for a number of years. An example of such a longstanding treatment method is the most common practice for treating an abscess, which generally includes the following steps: (1) Sterilizing the treatment site; (2) Applying a local anesthetic to the site; (3) Using a blade to incise the most fluctuant area of the abscess; (4) Using an instrument to express the purulent material contained within the tissue surrounding the abscess by breaking up the loculations; (5) Using forceps or a swab to repeatedly insert segments of one or more packing strips in order to fill the drained/irrigated abscess; (6) Covering the packed abscess with a bandage; (7) After approximately 48 hours, removing the packing strip(s) and repacking the abscess; and (8) Continuing the removal and repacking process until the site is no longer draining purulent material.

Steps 5, 7 and 8 listed above require use of one or more standard “shoestring-style” packing strips, which can require extensive, continuous packing in order to entirely fill a particularly sized abscess. Packing an abscess can be very painful for a patient and, because the above-described process requires repeated packing and unpacking of segments of the packing strip(s), the patient is forced to endure the associated pain a considerable number of times. A practical byproduct of this painful, segment-by-segment packing process is the likelihood that the care provider might pack the wound only to a suitable threshold rather than to completion due to the patient's discomfort. Improper packing procedure can result in outpatient therapy failure, thereby necessitating that the patient be admitted to the hospital. Moreover, because the above-described process requires an extended period of time for a physician to effectively complete, the associated medical costs for a patient are relatively high for what is otherwise a fairly routine procedure.

Other types of open wounds that often require packing include, but are not limited to: chronic non-healing ulcers; diabetic ulcers; superficial post-op surgical sites; bartholin gland cyst sites; and post-device (e.g., pacemakers) extraction openings.

Therefore, with the foregoing reasons in mind, there exists a need for an expanding article for packing an open wound, wherein the expanding article is selectively impregnable with one or more medicinal fluids.

### SUMMARY OF THE INVENTION

In accordance with one form of this invention, there is provided an article for packing irrigated wounds, the article including a main body being at least partially formed from a highly absorbent material, the main body being sized and configured for engaging the irrigated wound; the main body being structured and disposed for absorbing surrounding purulent fluid of the irrigated wound; wherein absorption of the surrounding purulent fluid in the irrigated wound causes the main body to continually expand until the outer wall of the main body comes into abutment with the inner wall of the irrigated wound; an elongated piece of material defining a wick extending from the main body, the wick being sized and configured for grasping during removal of the expanded main body from the irrigated wound; and the wick defining an evacuation channel extending between an opening at a distal end of the wick and a proximal end of the wick located within the main body, the wick being at least partially absorbent for permitting passage therethrough of the purulent fluid, and wherein the purulent fluid may be accessed and removed via the evacuation channel.

In accordance with another form of this invention, there is provided an article for packing irrigated wounds, the article including a main body being at least partially formed from a highly absorbent material, the main body being sized and configured for engaging the irrigated wound; the main body being structured and disposed for absorbing surrounding purulent fluid of the irrigated wound; wherein absorption of the surrounding purulent fluid in the irrigated wound causes the main body to continually expand until the outer wall of the main body comes into abutment with the inner wall of the irrigated wound; an elongated piece of material defining a wick extending from the main body, the wick being sized and configured for grasping during removal of the expanded main body from the irrigated wound; the wick defining an evacuation channel extending between an opening at a distal end of the wick and a proximal end of the wick located within the main body, the wick being at least partially absorbent for permitting passage therethrough of the purulent fluid, and wherein the purulent fluid may be accessed and removed via the evacuation channel; one or more porous regions formed in the main body, and each of the one or more porous regions being in liquid flow communication with the outer wall of the main body; and a fluid element that is impregnated in at least one of the one or more porous regions of the main body, the fluid element being exposed to the inner wall of the irrigated wound when the outer wall of the main body is in abutment with the inner wall of the irrigated wound.

In accordance with another form of this invention, there is provided a system for packing irrigated wounds, the system including a substrate; at least one article being selectively removable from the substrate, wherein the article includes a main body being at least partially formed from a highly absorbent material, the main body being sized and configured for engaging the irrigated wound; the main body being structured and disposed for absorbing surrounding purulent fluid of the irrigated wound; wherein absorption of the surrounding purulent fluid in the irrigated wound causes the main body to continually expand until the outer wall of the main body comes into abutment with the inner wall of the irrigated wound; an elongated piece of material defining a wick extending from the main body, the wick being sized and configured for grasping during removal of the expanded main body from the irrigated wound.

### BRIEF DESCRIPTION OF THE DRAWINGS

For a fuller understanding of the nature of the present invention, reference should be made to the following detailed description, taken in conjunction with the accompanying drawings in which:

FIG. 1 is a side elevational view illustrating the dermis layer, including the epidermis, dermis and hypodermis, muscle and bone;

FIG. 2 is a side elevational view illustrating a non-irrigated wound formed within the dermis layer;

FIG. 3 is a side elevational view of the wound packing article of the present invention according to one embodiment in an irrigated, open wound;

FIG. 4 is a side elevational view of the wound packing article according to the embodiment shown in FIG. 3 after it has expanded against the inner facing wall of the irrigated, open wound;

FIG. 5 is a side elevational view of the wound packing article of the present invention according to another embodiment in an irrigated, open wound;

FIG. 6 is a side elevational view of the wound packing article according to the embodiment shown in FIG. 5 after it has expanded against the inner facing wall of the irrigated, open wound;

FIG. 7 is a perspective view shown in partial cross-section, illustrating one embodiment of the wound packing article of the present invention, wherein an evacuation tube extends the length of the wick;

FIG. 8 is a side elevational view, shown in partial cross-section, of the wound packing article of according to the embodiment shown in FIG. 7 and illustrating the removal of fluid through the evacuation tube when the wound packing article is in an irrigated, open wound;

FIG. 9 is a perspective view of a packaging system for securing the wound packing articles prior to use; and

FIG. 10 is a perspective view illustrating removal of a wound packing article from the packaging system.

Like reference numerals refer to like reference parts throughout the several views of the drawings.

### DETAILED DESCRIPTION OF THE PREFERRED EMBODIMENT

Referring to the several views of the drawings, the impregnable and expanding wound packing article of the present invention for packing open wounds 100 is shown and is generally indicated as 10.

Referring initially to FIGS. 1-4, the wound packing article 10 includes a main body 12 and a wick 14 extending therefrom. The main body 12 is sized and configured for engaged receipt within an open wound 100, such as an irrigated abscess on the surface of a patient's skin. Different embodiments of the main body 12 may be formed having variously sized three-dimensional shapes (e.g., spherical, ovoidal, disk-shaped, etc.) and volumes. The wick 14 is an elongated piece of material that is secured to the main body 12 or, alternatively, formed integrally therefrom.

The main body 12 of the wound packing article 10 is at least partially made from one or more highly absorbent materials, such as polyurethane sponge material or compressed cotton gauze sponge material. The main body 12 may include an outer and/or inner layer of the absorbent material or, alternatively, may be made entirely from the one or more absorbent materials.

In a preferred embodiment, the main body 12 of the wound packing article 10 includes a component that is at least partially impregnable with a fluid element, such as a medicinal fluid (e.g., an antibiotic solution). The main body 12 is structured and disposed to emit the impregnated fluid element over a period of time when received within an open wound. In one embodiment, the emission of the impregnated element from the main body 12 is directly related to the absorption of bodily fluids by the main body 12.

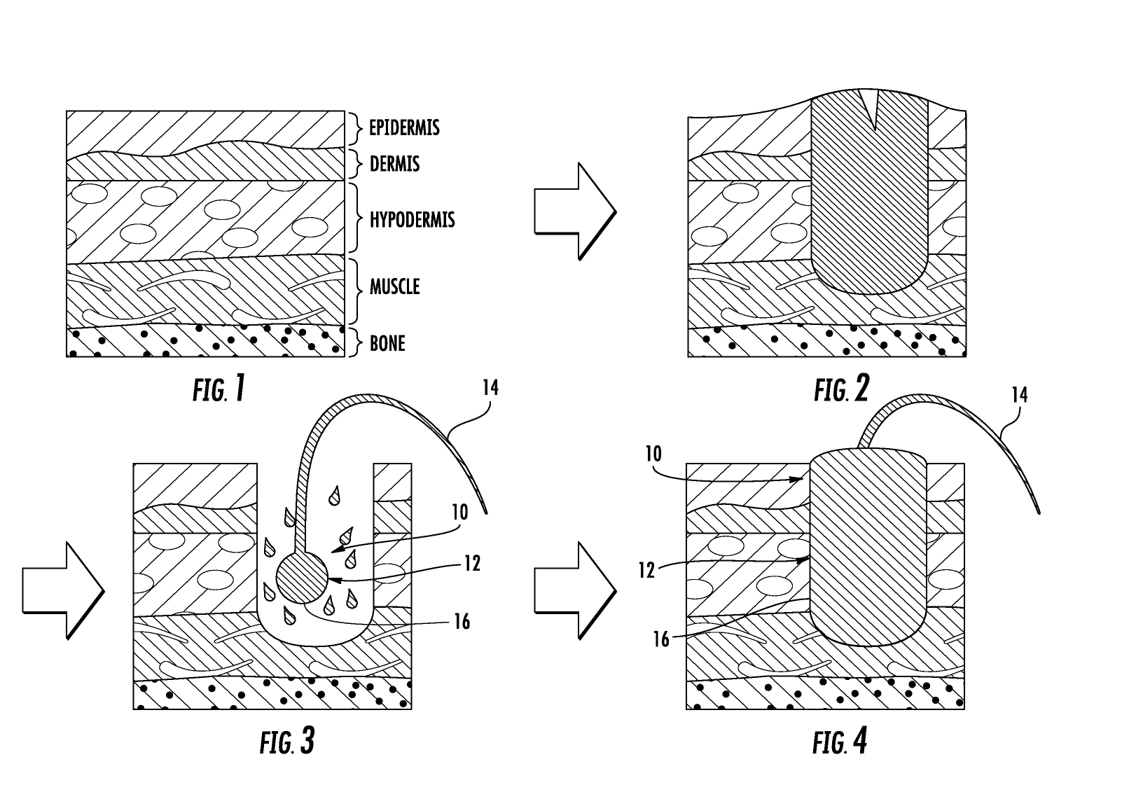

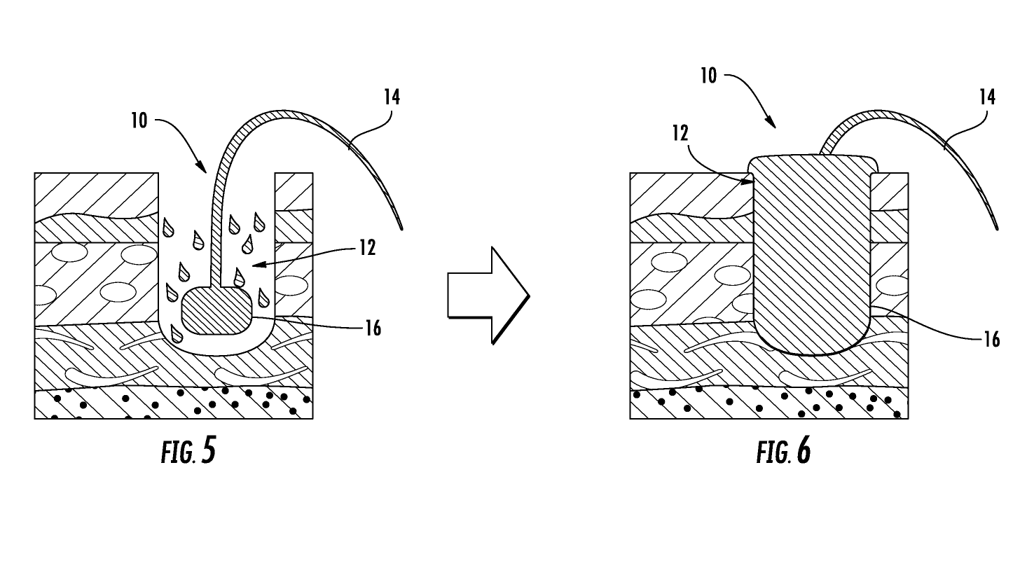

Referring specifically to FIGS. 3 and 4, the main body 12 of the wound packing article 10 is positioned within the irrigated, open wound 100 (e.g., irrigated abscess). A preferred method of introducing the main body 12 within the irrigated, open wound 100 is with forceps. FIG. 3 illustrates a first embodiment of the wound packing article 10, wherein the main body 12 is spherical. After the main body 12 is packed—which is a one step process—the wound is covered with a bandage. Over time, the wound cavity will fill with bodily fluids (e.g., purulent material) and the main body 12 absorbs the bodily fluids introduced thereto. In response to absorption of surrounding fluids, the main body continually expands until the outer wall of the main body 12 comes into abutment with the inner wall of the irrigated, open wound, as shown in FIG. 4. After approximately 48 hours, the expanded main body 12 is removed (one step process) from the irrigated, open wound 100 using the wick 14. The wound 100 may then be packed again with an unused, secondary wound packing article 10 if determined to be necessary by the care provider.

In a preferred embodiment, the outer wall 16 of the main body 12 is formed from a material having density sufficient for preventing surrounding tissue from growing into main body 12. In one embodiment, the gaps in the material forming the outer wall 16 are sufficiently narrow to prevent tissue growing therethrough.

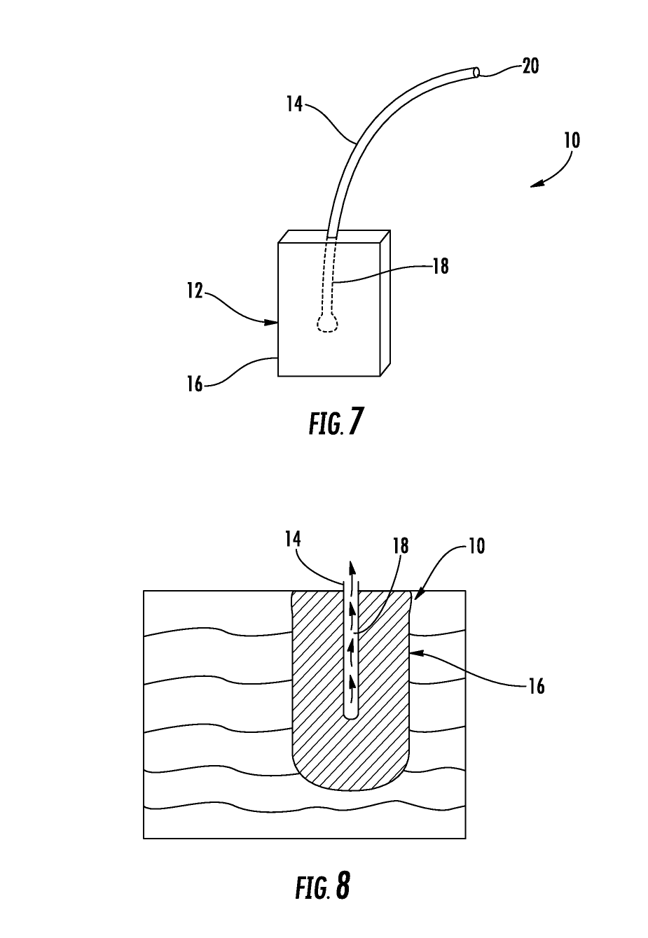

Referring to FIGS. 5 and 6, another embodiment of the wound packing article 10 is shown, wherein the main body 12 is disk-shaped. The packing, absorption, expansion progression of the disk-shaped main body 12 is the same as described above for the spherical main body 12. Alternatively shaped main bodies 12 are considered and may be manufactured to suit particularly shaped open wounds.

In one embodiment of the wound packing article 10, the same material is used to form one or more of the highly absorbent, expanding and outer wall 16 portions of the main body 12. In other embodiments, the wound packing article 10 may be made from open and closed cell packing material.

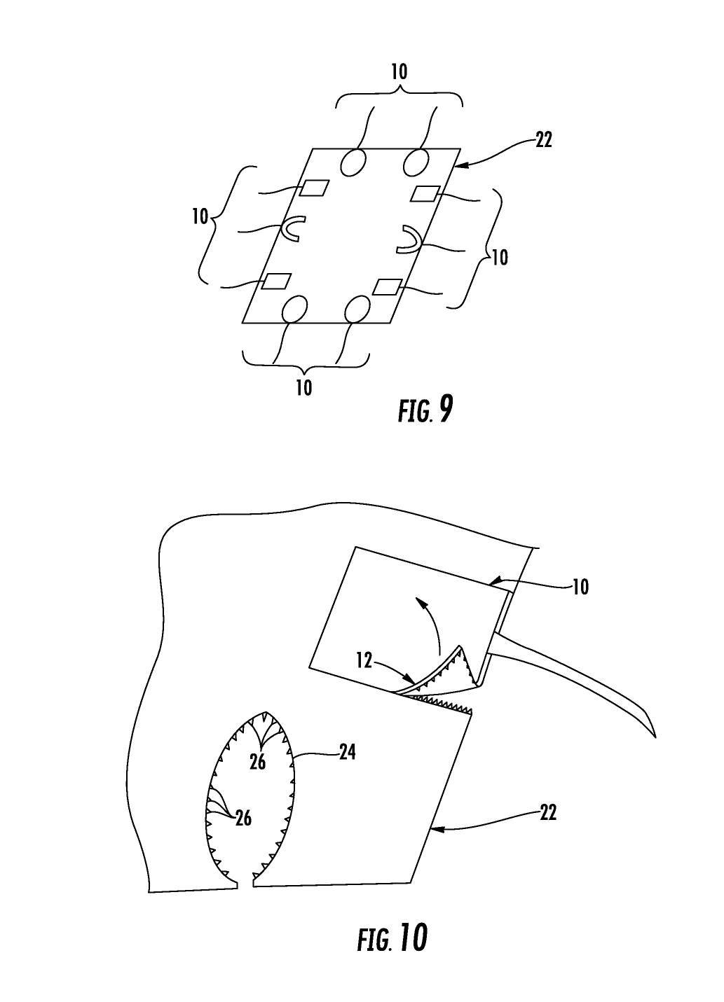

Referring to FIGS. 7 and 8, in one embodiment the wick 14 is formed to surround an evacuation channel 18 extending between an opening 20 at the top of the wick 14 and a termination point wherein at least a portion of the evacuation channel 18 enters beyond the location of where the wick 14 meets the main body 12. In one embodiment, the evacuation channel has a 4 mm diameter. Referring specifically to FIG. 8, the evacuation channel 18 permits drainage of the fluid absorbed by the main body 12 of the packing article 10 via passage of fluid therethrough. In this embodiment, the wick 14 is porous to permit passage of the fluid between the main body 12 and the evacuation channel 18. In one embodiment, the wick 14 only permits inward passage of fluid (i.e., from the main body 12 into the evacuation channel 18). The opening 20 on wick 14 may be sized to permit entry of a device (not pictured), such as a hypodermic needle, to draw the fluid outwards from the main body 12.

Referring to FIGS. 9 and 10, wound pack articles 10 may be manufactured as part of a packaging unit 22, the packaging unit 22 being defined by a substrate. As illustrated, each packaging unit 22 may include a variety of shapes of main bodies 12 of the packing articles 10. Alternatively, a packaging unit 22 can include a plurality of main bodies 12 having the same shape. Referring specifically to FIG. 10, the packing articles 10 are removable from the packaging unit 22. In one embodiment, the packing articles 10 are secured and selectively removable from openings 24. Each opening 24 includes perforations 26 where the packing article 10 meets the packaging unit 22 so that the packing article 10 can be easily separated, i.e., torn away, from the packaging unit 22 for use of the packing article 10 as disclosed above.

While the present invention has been shown and described in accordance with several preferred and practical embodiments, it is recognized that departures from the instant disclosure are contemplated within the spirit and scope of the present invention.

What is claimed is:

1. An article for packing irrigated wounds, said article comprising:
a main body being at least partially formed from a highly absorbent material, said main body being sized and configured for engaging the irrigated wound;
said main body being structured and disposed for absorbing surrounding purulent fluid of the irrigated wound;
wherein absorption of the surrounding purulent fluid in the irrigated wound causes the main body to continually expand until the outer wall of said main body comes into abutment with the inner wall of the irrigated wound;
an elongated piece of material defining a wick extending from the main body, said wick being sized and configured for grasping during removal of said expanded main body from the irrigated wound; and
said wick defining an evacuation channel extending between an opening at a distal end of said wick and a proximal end of said wick located within said main body, said wick being at least partially absorbent for permitting passage therethrough of the purulent fluid, and wherein the purulent fluid may be accessed and removed via said evacuation channel.

2. The article as recited in claim 1 wherein said highly absorbent material is a polyurethane sponge material.

3. The article as recited in claim 1 wherein said highly absorbent material is a compressed cotton gauze sponge material.

4. The article as recited in claim 1 wherein said main body is spherical-shaped.

5. The article as recited in claim 1 wherein said main body is disc-shaped.

6. The article as recited in claim 1 wherein said main body is ovoidal-shaped.

7. The article as recited in claim 1 wherein said evacuation channel is four (4) millimeters in diameter.

8. An article for packing irrigated wounds, said article comprising:
a main body being at least partially formed from a highly absorbent material, said main body being sized and configured for engaging the irrigated wound;
said main body being structured and disposed for absorbing surrounding purulent fluid of the irrigated wound;
wherein absorption of the surrounding purulent fluid in the irrigated wound causes the main body to continually expand until the outer wall of said main body comes into abutment with the inner wall of the irrigated wound;
a wick extending from the main body, said wick being sized and configured for grasping during removal of said expanded main body from the irrigated wound;
said wick defining an evacuation channel extending between an opening at a distal end of said wick and a proximal end of said wick located within said main body, said wick being at least partially absorbent for permitting passage therethrough of the purulent fluid, and wherein the purulent fluid may be accessed and removed via said evacuation channel;
one or more porous regions formed in said main body, and each of said one or more porous regions being in liquid flow communication with the outer wall of said main body; and
a fluid element that is impregnated in at least one of said one or more porous regions of said main body, said fluid element being exposed to the inner wall of the irrigated wound when the outer wall of said main body is in abutment with the inner wall of the irrigated wound.

9. The article as recited in claim 8 wherein said highly absorbent material is a polyurethane sponge material.

10. The article as recited in claim 8 wherein said highly absorbent material is a compressed cotton gauze sponge material.

11. The article as recited in claim 8 wherein said main body is spherical.

12. The article as recited in claim 8 wherein said main body is disc-shaped.

13. The article as recited in claim 8 wherein said main body is ovoidal-shaped.

14. The article as recited in claim 8 wherein said fluid element is medicinal fluid.

15. The article as recited in claim 14 wherein said fluid element is an antibiotic solution.

16. The article as recited in claim 8 wherein said evacuation channel is four (4) millimeters in diameter.

17. A system for packing irrigated wounds, said system comprising:
a substrate;
at least one article being selectively removable from said substrate, wherein said article comprises:
a main body being at least partially formed from a highly absorbent material, said main body being sized and configured for engaging the irrigated wound;
said main body being structured and disposed for absorbing surrounding purulent fluid of the irrigated wound;
wherein absorption of the surrounding purulent fluid in the irrigated wound causes the main body to continually expand until the outer wall of said main body comes into abutment with the inner wall of the irrigated wound; and
an elongated piece of material defining a wick extending from the main body, said wick being sized and configured for grasping during removal of said expanded main body from the irrigated wound.

18. The system for packing irrigated wounds as recited in claim 17, wherein said substrate includes at least one opening being defined by said substrate, said opening being sized to securely hold one of said at least one articles, and wherein said at least one article is secured to said substrate via perforations between said at least one article and said substrate.

19. The system for packing irrigated wounds as recited in claim 18 wherein said wick defines an evacuation channel extending between an opening at a distal end of said wick and a proximal end of said wick located within said main body, said wick being at least partially absorbent for permitting passage therethrough of the purulent fluid, and wherein the purulent fluid may be accessed and removed via said evacuation channel.

20. The system for packing irrigated wounds as recited in claim 18 further comprising:
one or more porous regions formed in said main body, and each of said one or more porous regions being in liquid flow communication with the outer wall of said main body; and
a fluid element that is impregnated in at least one of said one or more porous regions of said main body, said fluid element being exposed to the inner wall of the irrigated wound when the outer wall of said main body is in abutment with the inner wall of the irrigated wound.
